(19) United States
(12) Patent Application Publication
(10) Pub. No.: US 2014/0241995 A1
(43) Pub. Date: Aug. 28, 2014

(54) MICROPATCH FOR ASSESSING CHEMICAL CONTACT ALLERGY

(71) Applicant: HDS Ltd., Shambrook (GB)

(72) Inventors: David Basketter, Bedfordshire (GB); Ian White, Bedfordshire (GB); John McFadden, Bedfordshire (GB)

(73) Assignee: HDS Ltd., Shambrook (GB)

(21) Appl. No.: 14/129,802

(22) PCT Filed: Jun. 28, 2012
(86) PCT No.: PCT/EP2012/062579

§ 371 Date: Apr. 9, 2014

(30) Foreign Application Priority Data
Jun. 29, 2011 (GB) 1111037.6
Jan. 12, 2012 (GB) 1200440.4

(51) Int. Cl. A61K 49/00

(52) U.S. Cl. CPC A61K 49/0006
USPC 424/9.81

(57) ABSTRACT

The micropatch of the present invention provides a reliable and effective system of elicitation necessary for the diagnostic identification of allergy cases whilst substantially reducing the chances of sensitising subjects, the present invention employs a micropatch.

The invention relates to a micropatch for use in chemical contact allergy testing to identify individuals exhibiting contact allergy to one or more allergens.

Current screening methods for chemical contact allergy typically consist of applying allergens in petrolatum using an 8 mm diameter (0.5 cm²) aluminium chamber, an 8 mm×8 mm square plastic chamber, or a 10 mm×10 mm impregnated stamp. The allergens are part of a collection of common allergens (e.g., the European Baseline Series) which are usually applied to the upper back for 2 days.

When the allergens are removed the skin is read for any reaction at this time and again a further 1-5 days later using standardised criteria (e.g., ICDRG criteria) (Fregert S. Manual of Contact Dermatitis, 2^{nd} Edition Copenhagen, Munksgaard 1981).

A positive reaction indicates contact allergy to the allergen(s) in question.

The standard method for screening for hair dye allergy is with the allergen aromatic amine para-phenylenediamine (PPD). In European clinics typically between 2% and 5% of patients screened are positive for PPD allergy (Thyssen J P, White J M. Epidemiological data on consumer allergy to p-phenylenediamine. Contact Dermatitis 2008; 59: 327-3).

Patch testing enables the identification of agents to which a subject is allergic. This provides a substantial benefit to the subject, as once he is aware of which chemicals are causing an allergic reaction he can take steps to avoid the allergenic compounds in order to prevent allergic contact dermatitis. However, in common with other in vivo diagnostic procedures, patch testing poses potential harmful effects. A rare but significant adverse event associated with patch testing is active sensitization.

Active sensitization caused by the diagnostic patch test itself (ie. the patient becomes sensitised as a result of the actual diagnostic process) is a significant problem. It has been estimated that active sensitization occurs after approximately 1 in 600-1000 cases (White J M, Gilmour N J, Jeffries D et al. A general population from Thailand; incidence of common allergens with emphasis on para-phenylenediamine. Clin Exp Allergy 2007; 37 (12): 1848-53).

The frequency with which active sensitization occurs during PPD patch testing is disputed, some reports rate the incidence of active sensitization is as high as 1.5% (Devos S A, van der Valk P G. The risk of active sensitization to p-phenylenediamine. Contact Dermatitis 2001; 44: 273-275) whilst others report the rate of sensitization caused by the test to be less than 0.2% (Dawe S A, White I R, Rycroft R J G et al. Active sensitization to para-phenylenediamine and its relevance: a 10-year review. Contact Dermatitis 2004; 51: 96-97). Nevertheless, of all the allergens used in standard chemical contact allergy screening, PPD is generally regarded as the allergen most likely to cause active sensitization.

In an attempt to reduce the frequency with which sensitization occurs, testing at a reduced concentration has been attempted. However, this more than halves the rate of detection of allergic individuals, rendering the test useless as a screen for detecting hair dye allergy.

A need exists for an accurate and reliable means of identifying allergic individuals which does not expose subjects to the risk of becoming sensitised by the test itself.

More specifically a need exists for a patch test procedure which reliably identifies all subjects allergic to an agent, through controlled elicitation, but which does not induce active sensitization in any of the other, non-allergic subjects that are tested.

In a first aspect the invention relates to a micropatch for chemical contact allergy testing to identify individuals exhibiting contact allergy to one or more allergen having an application area of less than 0.5 cm².

In another aspect the invention relates to a micropatch wherein the micropatch is provided by a small chamber.

In another aspect the invention relates to a micropatch wherein the micropatch is provided by a stamp.

### DETAILED DESCRIPTION

Sensitization exposure to an allergen leads to the formation of a clone of lymphocytes which will react to the allergen on subsequent exposure. With regards to contact allergens such as hair dye chemicals, cutaneous exposure leads to transport to the local lymph node by ‘antigen presenting cells’ and the clone of T cells will be produced there.

Elicitation is a local inflammatory reaction to an allergen in an individual who is already sensitised to the allergen. By way of example elicitation by skin exposure to hair dye chemicals initiates a T cell mediated inflammatory response in the skin.

Contact Allergy is the existence of sensitization to chemicals such as hair dye.

Allergic contact dermatitis is the skin elicitation reaction to an allergen in a susceptible ie a person with contact allergy to the allergen.

Patch testing is a procedure for diagnosing allergic contact dermatitis. It exposes an individual to a small concentration of a contact allergen under occlusion to the skin in order to produce a controlled, limited elicitation reaction which will enable a diagnosis of contact allergy to be made.

Active sensitization is the uncommon but important adverse event where patch testing causes sensitization and subsequent contact allergy in an individual to the allergen which is being tested.

It is well established in the prior art that the critical factor in the induction of allergic sensitization (becoming allergic) to a single allergen following skin exposure is the dose per unit area (mg/cm²) (Kimber I, Dearman R J, Basketter D A, Ryan C A, Gerberick G F, Lalko J and Api A M. (2008) Dose metrics in the acquisition of skin sensitization: thresholds and importance of dose per unit area. Regulatory Toxicol Pharmacol 2008; 52: 39-45).

It does not matter whether the exposed area is 1 cm² or 10 cm², if the dose per unit area is the same then the chances of becoming sensitised are the same (Friedmann P S The relationship between exposure dose and response in induction and elicitation of contact hypersensitivity in humans. Br J Dermatol 2007; 157: 1093-1102).

The results reported in Friedmann are reproduced below:

Subjects were exposed to the strong allergen 2,4-dinitrochlorobenzene (DNCB) (approximately equivalent in strength to PPD).

TABLE 1
Effect of surface area dose and concentration (dose per unit area)
on sensitization with DNCB allergen

| | | | Sensitising dose | | | |
|---|---|---|---|---|---|---|
| | Application data | | | Concen- | | |
| | Diameter | | Total | tration | | % |
| Row | cm | Area cm² | (μg) | (μg/cm²) | Number | sensitised |
| 1 | 3 | 7.1 | 1000 | 142 | 24 | 100 |
| 2 | 3 | 7.1 | 500 | 71 | 40 | 100 |
| 3 | 3 | 7.1 | 250 | 35.4 | 30 | 83 |
| 4 | 3 | 7.1 | 125 | 17.1 | 30 | 63 |
| 5 | 3 | 7.1 | 62.5 | 8.8 | 24 | 8 |
| 6 | 1.5 | 1.8 | 62.5 | 35.4 | 7 | 86 |
| 7 | 2.1 | 3.5 | 58 | 16.4 | 22 | 55 |
| 8 | 3 | 7.1 | 116 | 16.4 | 34 | 50 |
| 9 | 4.25 | 14.2 | 232 | 16.4 | 15 | 66 |
| 10 | l cm paper | 0.8 | 30 | 38 | 28 | 93 |
| 11 | 3 mm paper | 0.08 | 3 | 38 | 15 | 26 |

The area of exposure in Rows 1 to 5 to DNCB is the same (7.1 cm²), illustrating the effect on sensitization rates of reducing the concentration of the sensitising dose applied to the same area of skin.

In the two highest concentrations (Rows 1 and 2) 142 and 71 μg/cm² (respectively) all subjects (100%) are sensitised. However, from row 3 to 5 where decreasing doses per unit area, (35.4, 17.1, and 8.8 μg/cm²) are administered the number of subjects sensitised is reduced from 83%, to 63% to only 8%.

Rows 3 and 6, demonstrate that comparable sensitization rates (83% and 85% respectively) result when the same dose per unit area (35.4 μg DNCB/cm²) is applied.

In rows 7-9 the application area ranges from 3.5, 7.1 to 14.2 cm² whilst the dose per unit area is kept constant. Again sensitization rates are equivalent (55%, 50% and 66%).

However when the surface area of exposure falls below 1 cm² the total dose becomes critical to sensitization frequency.

Row 10 discloses a surface area exposure of 0.8 cm² a total dose of 30 μg DNCB and a dose per unit area of 38 μg/cm². Row 11 has the same dose per unit area but only 1/10 surface area of 0.08 cm² and a 1/10 total dose of 3 μg DNCB. However the number of individuals sensitised has significantly fallen from 93% to 26%.

Thus Friedmann also demonstrates that the direct relationship between dose per unit area and sensitization frequency breaks down when the area of application is below 1 cm² and instead total dose becomes critical.

Friedmann interprets these results with respect to overall numbers of antigen presenting cells (Langerhans cells) in the skin. The mean density of Langerhans cells in the forearm skin is about 750 per mm², so an area 1 cm² contains about 59,000 Langerhans cells (Ford G P, Friedmann P S, White S I et al. Possible inhibitory mechanisms for contact sensitization by DNCB following UVB induced damage to Langerhans cells Br J Dermatol 1984; 111: 701-702). After application of a contact allergen upto 20% of these Langerhans cells migrate and are therefore involved in sensitization process (Cumberbatch M Clelland K Dearman R J et al Impact of cutaneous IL-10 on resident epidermal Langerhans cells and the development of polarised immune response. J Immunol 2005; 175: 43-50). Therefore, 6-1200 Langerhans cells are required for optimal sensitization to a contact allergen.

The inventors recognised that by reducing the area of exposure using a micropatch in an otherwise conventional skin patch test they would reduce the total number of Langerhans cells exposed to the sensitising agent and would reduce the risk of active sensitization.

They further recognised that a micropatch would deliver a safer patch testing option which would be much less likely to induce inadvertent sensitization when compared to conventional patch testing methods.

Following this realisation the inventors readily identified several studies which supported this approach, Schnitzer A. Beitrag zur Frage des Mechanism der Sensibiliserung Dermatologica 1942; 85: 339-347 first established, using the contact allergen DNCB on guinea pigs, that sensitization to contact allergens was dependant upon the concentration and not on the exposed area. Similarly Magnusson B, Kligman A M. Induction of hypersensitivity in: Allergic contact dermatitis in the guinea pig. Identifications of contact allergens. Springfield Thomas C C 1970; 44-7 substantiated the same principle using, again, guinea pigs.

Conventional patch testing has always been conducted using patches of at least 0.5cm² as it is a well established convention in the field that to use smaller patch areas would reduce elicitation rates and thus the reliability of the patch test.

However (Fischer L A, Menné T, Johansen J D. Dose per unit area—a study of elicitation of nickel allergy. Contact Dermatitis 2007; 56: 255-261) investigates the effect of area of exposure on elicitation rates.

20 subjects with proven allergy to nickel (having a positive patch test to standard 5% nickel sulphate) were tested with low concentrations of nickel sulphate on one side of their back whilst the other side was tested with concentrations ten times higher. As per standard patch testing, the patches were applied for 2 days then read at day 3 or 4 and day 7. The results are shown with table 2 below:

TABLE 2
Patch test/elicitation reactions to nickel. The dose per area and the
total dose applied in the patch test

| | | | | Total | | | |
|---|---|---|---|---|---|---|---|
| | | Concen- | | Ni | | | |
| Num- | Area | tration | Ni/cm² | dose | Number | Score | Mean |
| ber | cm² | (%) | (μg) | (μg) | reacting | range | score |
| 1 | 0.50 | 0.08 | 6.6 | 3.3 | 6/20 | 0-5 | 0.5 |
| 2 | 1.13 | 0.08 | 6.6 | 7.5 | 8/20 | 0-3 | 0.5 |
| 3 | 0.50 | 0.20 | 15 | 7.5 | 10/20 | 0-5 | 0.8 |
| 4 | 1.13 | 0.20 | 15 | 17 | 15/20 | 0-7 | 2.1 |
| 5 | 0.50 | 0.80 | 66.4 | 33.2 | 17/20 | 0-7 | 3.9 |
| 6 | 1.13 | 0.80 | 66.4 | 75 | 18/20 | 0-8 | 4.6 |
| 7 | 0.50 | 1.90 | 150 | 75 | 19/20 | 0-8 | 5.0 |
| 8 | 1.13 | 1.90 | 150 | 169.5 | 19/20 | 0-8 | 5.3 |

In Rows 1 to 4 the concentration of Ni applied (0.08 and 0.2%) is very low when compared to conventional patch testing which uses a concentration of 5% which likely accounts for the difference in elicitation/positive patch test results (6 vs 8, 10 vs 15).

However when the dose was raised (to 0.80 and 1.9%) in rows 5 to 8 there was no appreciable difference in elicitation rates (17 vs 18, 19 vs 19) even when the area of exposure is reduced from 1.13 to 0.5 cm².

These data support the inventors' realisation that elicitation and hence the ability of a patch test to reliably identify allergic individuals is not dependant on patch size, when the area of exposure is less than 1 cm². Which taken in conjunction with the recognition that by reducing patch sizes the risk of active sensitization is significantly reduced, resulted in the development of a micropatch of less than 0.5 cm².

15 volunteers with a positive history of contact allergy to p-phenylenediamine were recruited. Contact allergy to p-phenylenediamine was determined based on their response to a standard 48 h, 8 mm diagnostic patch test with 1% p-phenylenediamine in petrolatum. According to their known sensitivity, these volunteers were tested with either 0.1% or 1.0% p-phenylenediamine in petrolatum, under occlusion, with treatment diameters of 8, 4, 3, and 2 mm. Reactions at 48 h, 72 h and/or 96 h were recorded using the Internationally accepted dermatology grading scale of N, ?+, +, ++. The responses were transposed into numerical values (0-3 respectively) and summated. The summated scores are presented below.

It is evident that reduced patch test diameter had no detectable impact on the reaction intensity observed.

These data demonstrate that the micropatch of the present invention, provides a reliable and effective system of elicitation necessary for the accurate identification of allergy cases, whilst substantially reducing the chances of sensitising subjects.

The present invention provides a variety of micropatches of different types being chambers or impregnated stamps each having an application area of less than 0.5 cm². Preferably the micropatch is provided by a small chamber or ‘stamp’ as is well known in the art.

Optionally the micropatch has an application area of less than 0.45 cm².
Optionally the micropatch has an application area of less than 0.4 cm².
Optionally the micropatch has an application area of less than 0.35 cm².
Optionally the micropatch has an application area of less than 0.3 cm².
Optionally the micropatch has an application area of less than 0.25 cm².
Optionally the micropatch has an application area of less than 0.2 cm².
Optionally the micropatch has an application area of less than 0.15 cm².
Optionally the micropatch has an application area of less than 0.1 cm².
Optionally the micropatch has an application area of less than 0.05 cm².
Optionally the micropatch has an application area of less than 0.04 cm².
Optionally the micropatch has an application area of less than 0.03 cm².
Optionally the micropatch has an application area of less than 0.02 cm².
Optionally the micropatch has an application area of less than 0.01 cm².
Optionally the micropatch has an application area of less than 0.005 cm².

Preferably the micropatch is used to apply allergens in the existing diagnostic patch test concentrations. Optionally the concentration and/or the vehicle employed can be varied as necessary.

The micropatch may be applied to any suitable site on the body, more preferably the micropatch may be applied to the upper arm; thus avoiding any potential, theoretical enhancement of sensitization risk caused by lymphatic drainage of other allergens, as may occur when multiple patches are applied to the back.

1. A micropatch for chemical contact allergy testing to identify individuals exhibiting contact allergy to one or more allergen having an application area of less than 0.5 cm2.

2. A micropatch as claimed in claim 1 wherein the micropatch is provided by a small chamber.

3. A micropatch as claimed in claim 1 wherein the micropatch is provided by a stamp.

4. A micropatch as claimed in claim 1 having an application area of less than 0.4 cm2.

5. A micropatch as claimed in claim 1 having an application area of less than 0.3 cm.

6. A micropatch as claimed in claim 1 having an application area of less than 0.2 cm.

7. A micropatch as claimed in claim 1 having an application area of less than 0.1 cm.
